(19) United States
(12) Patent Application Publication
(10) Pub. No.: US 2026/0014288 A1
(43) Pub. Date: Jan. 15, 2026

(54) RIGID STERILIZATION SYSTEM FOR STERILIZATION

(71) Applicant: Turbett Surgical, Inc., Henrietta, NY (US)

(72) Inventor: Robert E. Turbett, Penfield, NY (US)

(21) Appl. No.: 18/994,163

(22) PCT Filed: Jul. 24, 2023
(86) PCT No.: PCT/US23/28472

§ 371 Date: Jan. 14, 2025

Related U.S. Application Data
(60) Provisional application No. 63/391,431, filed on Jul. 22, 2022.

(51) Int. Cl. A61L 2/26

(52) U.S. Cl. CPC A61L 2/26; A61L 2202/121; A61L 2202/123; A61L 2202/16

(57) ABSTRACT

A rigid sterilization system for use in a sterilizer for the sterilization of objects requiring sterilization, the system comprising a base formed of a material adapted to withstand exposure to a sterilizing agent, the base having (i) a support surface having a portion configured to support the objects requiring sterilization, and (ii) a first sealing surface, a cover having a first vent port and a first filter overlying the first vent port, the cover comprising a rigid material configured to hold a selected shape, wherein the lower portion of the rigid cover is configured to form a sealed interface with the base, and a continuous seal between the first sealing surface of the base and the cover, the continuous seal forming a continuous sealed interface between the base and the cover.

### BACKGROUND OF THE INVENTION

### Field of the Invention

The present invention relates to a sterilization system and method for sterilizing surgical instruments and the like and more particularly to a rigid sterilization system for sterilization comprising a base and a rigid cover and a method of using the same.

### DESCRIPTION OF RELATED ART

Cleaning and sterilizing surgical instruments, laboratory supplies, and medical devices are important in many fields including, but not limited to, scientific research, veterinary medicine, and human medicine. Surgical instruments, for example, must be provided in a sterile condition to be able to be used in surgery. To reduce the risk of introducing harmful microorganisms to a patient or in scientific research, several steps are typically followed. First, the objects may be decontaminated and pre-cleaned. Next, the objects are cleaned, rinsed and dried. Following a thorough cleaning, the objects are sterilized. Sterilization is a term referring to any process that eliminates, removes, deactivates, or kills bacteria or other types of living microorganisms, including transmissible agents (such as fungi, bacteria, viruses, or spore forms) present on a surface, or contained in a fluid, or in medication, or in a compound such as biological culture media. Sterilization can be achieved by applying heat, steam, chemicals, such as using ethylene oxide (EO), irradiation, high pressure, and filtration or combinations thereof. In general, surgical instruments, laboratory supplies, and medical devices must be sterilized to a high sterility assurance level before use on a body. Examples of such instruments include scalpels, hypodermic needles, endoscopes, and implantable medical devices (IMD), such as artificial pacemakers.

A widely used method for heat sterilization is the autoclave, sometimes referred to as a converter. Autoclaves commonly use steam heated to 121-134° C. To achieve a degree of sterility, a holding time of at least 15 minutes at 121° C. at 100 kPA, or 3 minutes at 134° C. at 100 kPa is required. Additional sterilizing time is usually required for liquids and instruments packed in layers of cloth, as they may take longer to reach the required temperature.

A method of sterilization involves wrapping instruments, supplies, medical devices, or trays containing such objects, with a packaging material to allow sterilization of the contents of the tray, and to maintain the sterility of the tray contents until the wrap is opened. Traditionally, instruments that need to be reprocessed are provided in vented trays, allowing the sterilizing agent to flow through the tray, and sterilizing all the surfaces. In order to maintain sterility after removal from the sterilizer, the trays are wrapped in a material that allows the sterilizing agent to pass through, but protecting the inner tray from microorganisms until use. A multitude of packaging materials can be used for sterilization, including woven fabrics, which may be made of cotton, a cotton-polyester blend, or synthetic blend, or nonwoven materials, which may be made of plastic polymers, cellulose fibers, or washed paper pulp bonded under pressure into sheets. Wrapping instruments, supplies, medical devices, or trays containing such objects, in packaging material, including but not limited to wraps sometimes results in the packaging material tearing or puncturing, in which case the sterilization process must be repeated with new packaging material. Further, wrapping instruments in this manner provides a significant surface area to inspect after running the sterilization cycle of a sterilizer. Single instruments can be individually sterilized in a peel pouch. The pouch maintains the sterility of the instrument after the sterilization process is complete by protecting it from the contaminants in the environment. Peel pouches, however, are not efficient as multiple tools cannot be sterilized within the same peel pouch. Further, peel pouches are to be loaded into a sterilizer on its side to promote air removal and penetration of the sterilant as well as to discourage moisture retention. Peel pouches cannot include implants having several component parts. These component parts would need to be packaged in separate peel pouches before sterilizing, typically by gamma irradiation processing.

Alternatively, the trays may be placed in a sterilization container utilizing filters that act as the wrap does, allowing the sterilizing agent in, but blocking microorganisms afterwards. The filter also allows the escape of the sterilizing agent or its byproducts. Current rigid container systems include a tub-like base having a bottom portion and sidewalls extending upwardly from the bottom portion. This tub-like base is configured to receive a tray of instruments. A problem with rigid-containers having a tub-like base is that the sidewalls of the base interfere with access to the instruments. Further, there is risk of contamination when reaching into the base to access the objects contained inside.

Another method of sterilization involves passing steam through a cabinet. For effective sterilization, steam needs to penetrate a cabinet load uniformly. Accordingly, the cabinet must not be overcrowded, and the lids of bottles and containers must be left ajar. During the initial heating of the chamber, residual air must be removed. Indicators should be placed in the most difficult places for the steam to reach to ensure that steam actually penetrates there.

A filter is typically placed over the vent to keep particles or extraneous materials from entering the cabinet before, during or after the sterilizing process. Once the sterilizing process is completed the filter needs to be removed and inspected by medical professionals to verify the integrity of the sterilizing process was maintained. If it is discovered during inspection that the filter did not remain intact, the sterilizing process has to be repeated with a new filter.

### BRIEF SUMMARY OF THE INVENTION

A first exemplary embodiment of the present disclosure provides a rigid sterilization system for an object requiring sterilization in a sterilizer, the rigid sterilization system comprising a base having a support surface configured to support an object, a rigid cover having a main body and a lower portion, the main body comprising a top panel and side panels extending from the top panel, wherein the top panel and side panels comprise a rigid material configured to hold a selected shape, wherein the rigid cover and the base define a volume encompassing the object requiring sterilization; and a sealing interface between the lower portion of the rigid cover and the base, the sealing interface configured to form a continuous seal between the lower portion of the rigid cover and a first sealing surface.

The rigid cover may further comprise a first vent port and a first filter overlying the first vent port and forming a sealed interface with an adjacent portion of the rigid cover. In one configuration, the base is a substantially flat surface. In one configuration, the base includes a rack system for holding the object. The base includes, in one configuration, a plurality of sides forming a dependent skirt, and wherein the first sealing surface is located on the dependent skirt. A gasket is between the lower portion of the rigid cover and the first sealing surface. In one configuration, the first sealing surface is located on the support surface and the first sealing surface further comprises a gasket within the first sealing surface on the support surface. In one configuration, the gasket is affixed to the lower portion of the rigid cover. The top panel, in one configuration, includes a window for viewing contents within the rigid sterilization system. In one configuration, the base is an operating room back table. The rigid cover can be made of heavy gauge aluminum in one configuration, or medical grade plastic material in another confirmation. In one configuration, a clamp releasably secures the rigid cover to the base. In one configuration, the filter is overlying the vent port. The rigid container system further comprises, in one configuration, a gasket surrounding the first filter, the first filter and gasket forming a filter cartridge, wherein the filter cartridge is removably secured to the main body. The first vent port is located on at least one of the top panel or one of the side panels. The lower portion of the rigid cover, in one configuration, overlies the first sealing surface of the base to form the sealed interface between the lower portion of the rigid cover and the base. In one configuration, the base further comprises a groove for receiving a gasket, wherein the lower portion of the rigid cover further comprises an edge, and wherein the edge engages the gasket within the groove for forming a perimetric seal between the edge and the base. The base, in one configuration, may further be configured to affix to a transport cart.

Another exemplary embodiment of the present disclosure provides a rigid sterilization system for an object requiring sterilization in a sterilizer, the rigid sterilization system comprising a base having a support surface configured to support an object, a rigid cover having a main body and a lower portion, the main body comprising a top panel and side panels extending from the top panel, wherein the top panel and side panels comprise a rigid material configured to hold a selected shape, wherein the rigid cover and the base define a volume encompassing the object requiring sterilization, and a sealing interface between the lower portion of the rigid cover and the base, the sealing interface configured to form a continuous seal between the lower portion of the rigid cover and a first sealing surface. In this configuration, a plurality of bases, each base having the rigid cover and the continuous seal between the lower portion of the rigid cover and the first sealing surface are provided, and, a rack having a plurality of shelves, each of the plurality of shelves configured to hold one of the plurality of bases having the cover after sterilization in a sterilizer.

A third exemplary embodiment of the present disclosure provides a rigid sterilization system for use in a sterilizer for the sterilization of objects requiring sterilization, the system comprising a base formed of a material adapted to withstand exposure to a sterilizing agent, the base having (i) a support surface having a portion configured to support the objects requiring sterilization, and (ii) a first sealing surface, a cover having a first vent port and a first filter overlying the first vent port, the cover comprising a rigid material configured to hold a selected shape, wherein the lower portion of the rigid cover is configured to form a sealed interface with the base, and a continuous seal between the first sealing surface of the base and the cover, the continuous seal forming a continuous sealed interface between the base and the cover. In one configuration, the support surface of the rigid base is substantially flat. The rigid sterilization system may further comprise a gasket between the lower portion of the rigid cover and the first sealing surface. The base may further comprise a plurality of sides forming a dependent skirt, and wherein the first sealing surface is located on the dependent skirt, and wherein the gasket is between the lower portion of the rigid cover and the dependent skirt. In one configuration, the first sealing surface is located on the support surface and wherein the gasket is located on one of the first sealing surface and the lower portion of the rigid cover. The base, in one configuration is affixed to a transport cart.

Another exemplary embodiment of the present disclosure provides a rigid sterilization system for use in a sterilizer for the sterilization of objects requiring sterilization, the system comprising a base formed of a material adapted to withstand exposure to a sterilizing agent, the base having (i) a support surface having a portion configured to support the objects requiring sterilization, and (ii) a first sealing surface, a cover having a first vent port and a first filter overlying the first vent port, the cover comprising a rigid material configured to hold a selected shape, wherein the lower portion of the rigid cover is configured to form a sealed interface with the base, and a continuous seal between the first sealing surface of the base and the cover, the continuous seal forming a continuous sealed interface between the base and the cover. In this configuration, a plurality of bases, each base having the cover and the continuous seal between the lower portion of the cover and the first sealing surface are provided, and, a rack having a plurality of shelves, each of the plurality of shelves configured to hold one of the plurality of bases having the cover after sterilization in a sterilizer.

### BRIEF DESCRIPTION OF THE SEVERAL VIEWS OF THE DRAWING(S)

FIG. 1 is a perspective view of an exemplary embodiment of the present disclosure rigid sterilization system in an unassembled position.

FIG. 2 is a perspective view of another exemplary embodiment of the present disclosure rigid sterilization system in an unassembled position.

FIG. 3 is a perspective view of the rigid sterilization system of FIG. 1 in an assembled position.

FIG. 4 is a perspective view of another exemplary embodiment of the present disclosure rigid sterilization system in an assembled position.

FIG. 5 is perspective view of another exemplary embodiment of the present disclosure rigid sterilization system in an unassembled position.

FIG. 6 is perspective view of another exemplary embodiment of the present disclosure rigid sterilization system in an unassembled position.

FIG. 7 is perspective view of yet another exemplary embodiment of the present disclosure rigid sterilization system in an unassembled position.

FIG. 8 is a partial cross-sectional view of the present disclosure rigid sterilization system taken from lines 8-8 of FIG. 3.

FIG. 9 is a perspective view of an exemplary embodiment of a transport cart having the present disclosure rigid sterilization system.

FIG. 10 is a perspective view of an exemplary embodiment of a holding rack for containing at least one of the present disclosure rigid sterilization systems.

### DETAILED DESCRIPTION OF THE INVENTION

It should be appreciated that the same reference numbers appearing in different figures identify the same structural elements of the present invention. While the description of the present invention includes what is currently considered to be the preferred configurations, it should be appreciated that the present invention is not limited to such configurations. Moreover, it should be appreciated that the present invention is not limited to the particular methodology, materials and modifications described herein and that the terminology used herein is not intended to limit the scope of the present invention. The scope of the present invention is therefore to be determined solely by the appended claims.

The invention pertains to a sterilizing system to provide significant vent to volume ratios for purposes of sterilizing instruments, medical devices, and the like during sterilization in a sterilizer. The term sterilizer includes, but is not limited to, a housing or device defining an interior retaining the sterilization system and in which a controlled environment is created to impart a desired sterilization. Sterilizers include autoclaves; hot air ovens; ethylene oxide; low temperature steam and formaldehyde; sporicidal chemicals; irradiation; chlorine dioxide (CD) gas sterilization; hydrogen peroxide; vaporized hydrogen peroxide; hydrogen peroxide plasma; electron beam and gas plasma devices.

By utilizing a base, such as a table, tray with a rack component or a plurality of trays with rack components, as a base component, one or more instruments may be placed directly on it. When a base, such as an operating room back table or tray or trays, is used, there is typically no need to transfer the instruments from a container onto a back table for use in the operating room, as the back table is integral to the sterilizing wrap system and thus, the instruments are already organized on the table and presentable after sterilization. Further, the instruments are easily accessible in that base sidewalls are not present to interfere with the removal of instruments.

By “operating room back table” or “back table” it is meant to generally refer to a table used in the operating room for holding surgical instruments. The operating room back table is sometimes known as an instrument table or a work table. The back table can be made of stainless steel, plastic, or other rigid materials, and can be any shape or size. Typically, the back table is a rectangular shaped table having legs and/or a pedestal which may or may not be adjustable and/or removable.

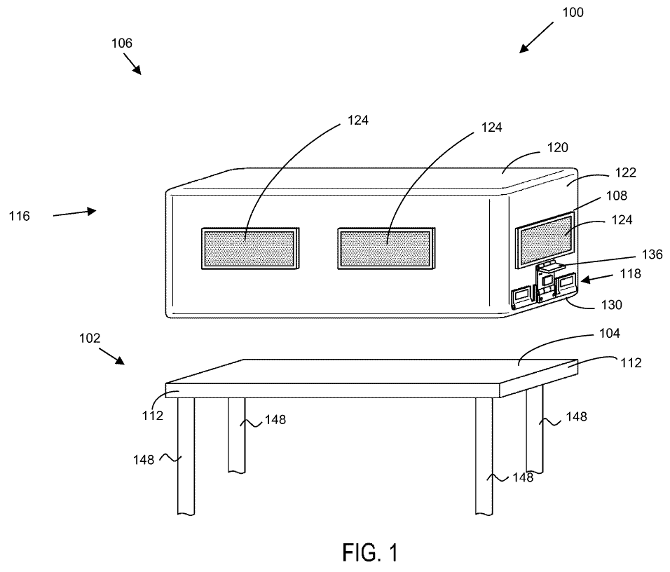

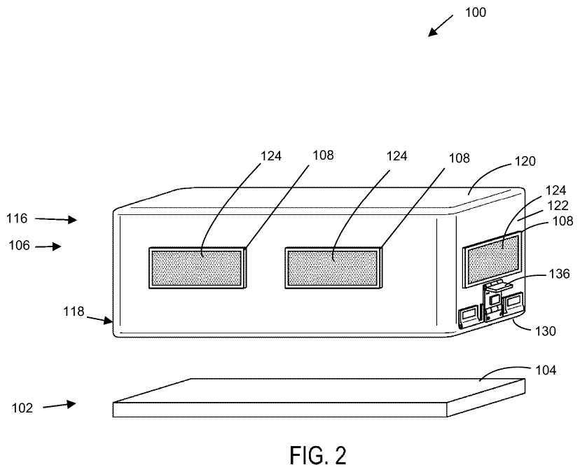

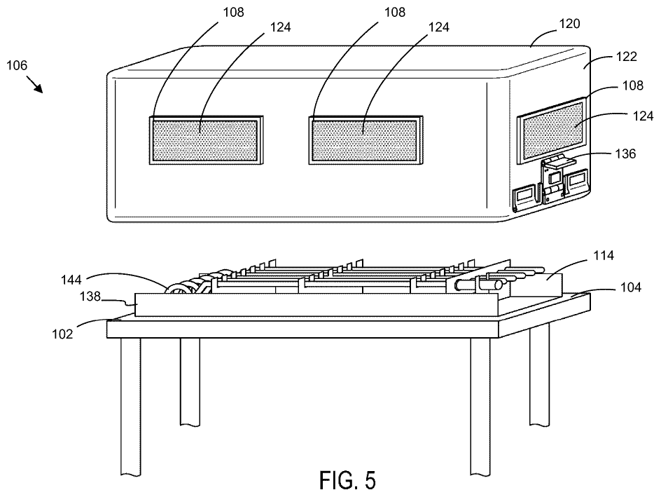

As shown in FIGS. 1-9, a rigid sterilization system 100 for an object requiring sterilization in a sterilizer comprises a rigid cover 106 and a base 102. In one configuration, the base 102 is an operation room back table having a support surface 104 for supporting at least one object 144 (as shown in FIG. 5) requiring sterilization by a sterilizer and legs 148. Objects 144 requiring sterilization may include, but are not limited to surgical tools and instruments, implants, laboratory supplies, and medical devices. The base 102 may be detachable from legs 148 or a pedestal (not shown). As shown in FIG. 2, the base 102 alternatively does not include legs. In one example embodiment, the base 102 is approximately 24 inches in length and 22.5 inches in width. However, the size of the base can vary. For example, the length of the base in certain configurations can range between 12 inches to 36 inches. The base 102 may be any shape and size for providing a support surface 104. For example, although base 102 is shown in a rectangular configuration, the base 102 may be any shape, including, but not limited to, circular, oval, square, and elliptical. The base 102 in this configuration is rigid and may be a table, sheet, plate, or any other type of rigid, supportive base.

In one configuration, the base 102 includes sidewalls 112. In another configuration, the base 102 includes a dependent skirt (not shown). Additionally, or alternatively, the base 102 may also include a flange or sidewalls 138 along the perimeter edge of the support surface 104 for retaining the objects 144 on the support surface 104, especially during transportation when objects may otherwise shift (for example, see FIG. 5). In one configuration, the base 102 is substantially flat. Alternatively, or additionally, the base 102 may include object separators 114 (as shown in FIG. 5), for example, rack, dividers, shelves, or sectioned portions for maintaining objects 144 on particular sections of the base 102. The object separators 114 may be affixed to the base 102. By “affixed” it is meant that the object separators 114 are one of permanently affixed, integral or removably affixed to the base 102. In one configuration, the base 102 supports a tray (not shown). The base 102 may further include additional fasteners, including but not limited to Velcro, magnets, and the like to releaseably hold the objects 144 in place. The base 102 may include a laser outline of at least some objects 144 identifying a preferred location for particular objects. Other features for organizing and maintaining objects 144 on the base 102 are intended to be included within the scope of the invention as claimed.

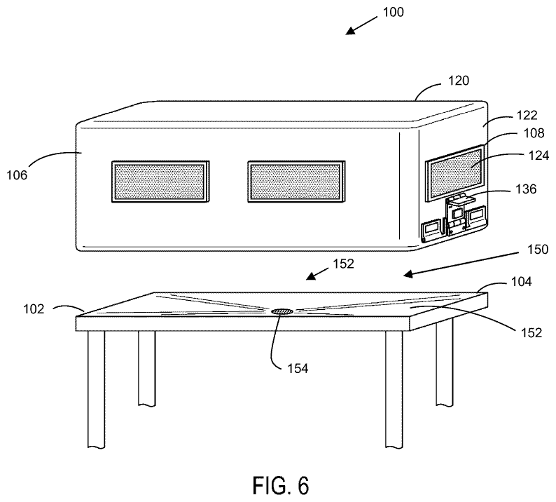

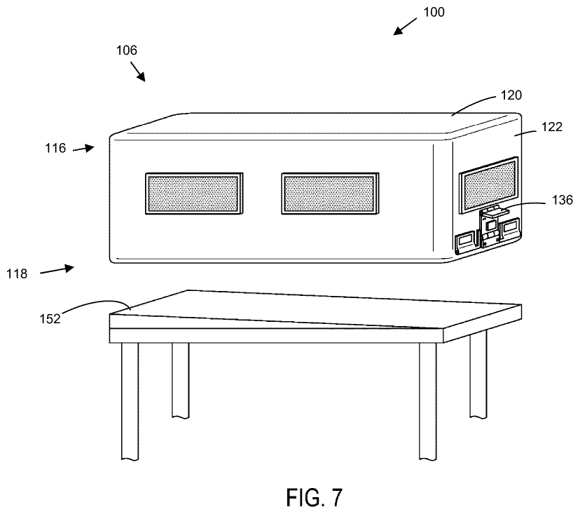

As shown in FIG. 6, in some configurations, the base 102 may include a drain system 150 for draining sterilizing agent and/or liquid condensate formed during the sterilization process. The base 102 may include, for example, a sloped surface 152, for example, as shown in either FIG. 6 or FIG. 7, to direct drainage to a predetermined area. The sloped surface 152 may be of any slope and may be a straight line (angular) or curved. In one configuration, the base 102 includes at least one drainage well having a downwardly sloping surface 152 arrange to direct condensate to a drain aperture 154. In one configuration, the drain aperture 154 is approximately 0.5 to 5 inches in diameter. In another configuration, the drain aperture 154 is approximately 2-3 inches in diameter. The drain aperture 154 may be any shape and size that allows liquid condensate or liquid sterilant to drain from the surface of the base 102. The drain aperture may further include a drain check valve and/or at least one filter. The surface 152 may be frusto-conical shaped to further direct sterilizing agent and condensate to the drain aperture 154. Alternatively, the drain system 150 may include a continuous decline directing sterilizing agent and/or liquid condensate to a drain well or aperture 152 located proximate one side of the base 102. It should be appreciated that the drain well or aperture 154 or a plurality of drain wells and/or apertures 152 may be positioned anywhere on the support surface 104 of the base 102, with the support surface 104 configured to permit drainage thereto. In yet another configuration, the drain system 150 may include a sloped surface or decline without a drain well or aperture 152, which directs sterilizing agent and/or liquid condensate to an edge of the support surface 104 of the base 10. The drain system 150 many include a filter or filters within the aperture 154. It should be appreciated by those having ordinary skill in the art that other drainage systems that allow for sterilizing agent and/or liquid condensate to drain from the support surface 104 of the base 102 are possible and these modifications are intended to be included within the scope of the claims. The base 102 may further include a holder to maintain the objects 144 above the drain system 150. The holder may be, for example, a plate having perforations, apertures, or wells, or a tray configured to be suspended above the base 102, among other things. The holder may be integral to the base 102 or removeable from the base 102. In one exemplary embodiment, the holder is the object separator 114 as shown in FIG. 5. It should be appreciated that there are many configurations known in the art to hold instruments, and in certain embodiments hold the instruments above a drain system, and these configurations are intended to be included within the spirit and scope of the claims.

As shown in FIGS. 1-7, the rigid sterilization system 100 further includes a rigid cover 106 having a main body portion 116 and a lower portion 118 having a lower edge 130. The rigid cover 106 is comprised of a rigid material configured to substantially hold a selected shape. In one example embodiment, the rigid cover 106 comprises a rigid materials configured to substantially hold a selected shape. For example, the rigid cover 106 may comprise an impact resistant medical grade plastic material. Alternatively, the rigid cover 106 may comprise a heavy gauge aluminum. In yet another exemplary embodiment, the rigid cover 106 comprises stainless steel. The base 102 may comprise the same material as the rigid cover 106. The rigid cover 106 and the base 102 when sealed provide a continuous barrier to bacteria or other types of living microorganisms, including transmissible agents (such as fungi, bacteria, viruses, or spore forms). The main body portion 116 of the rigid cover 106 comprises a top panel 120 and side panels 122. The lower portion 118 of the rigid cover 106 is configured to seal to the base 102. In one configuration, a sealing interface between the lower portion 118 of the rigid cover 106 and the base 102 forms a seal between the lower portion 118 and the base 106. In one configuration, the seal is a continuous seal.

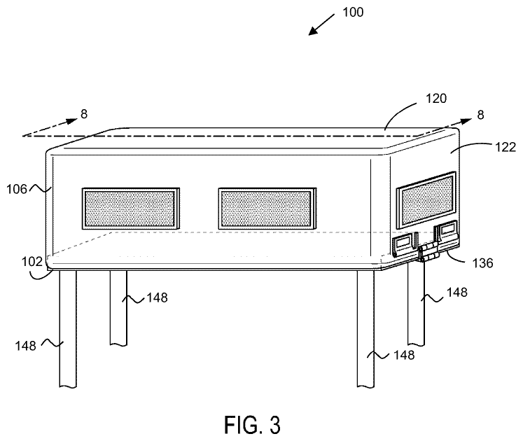

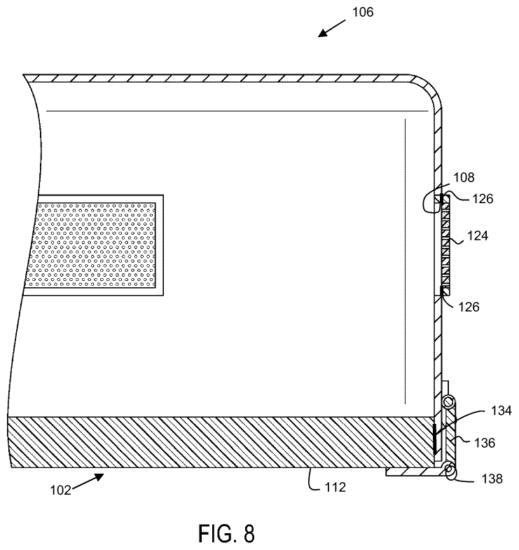

In an exemplary embodiment, as shown in FIGS. 3 and 8, the base 102 includes sides 112, for example, which includes a sealing surface. In one configuration, the base 102 includes a dependent skirt (not shown). Either or both of the rigid cover 106 and the sides 112 may include a gasket 134. In a configuration, the gasket 134 is affixed to either or both of the rigid cover 105 and the sides 112. By “affixed” it is meant that the gasket 134 is permanently affixed, integral or releasably affixed to either or both of the rigid cover 106 or sides 112. The gasket 134 forms a sealed interface with a confronting surface, such as the lower portion 118 of the rigid cover 106 and/or the base 102, and is able to maintain its integrity during and after undergoing a sterilization cycle. The gasket 134 may be made of any type of material that is both substantially rigid and is able to maintain its integrity during and after undergoing a sterilization cycle. Exemplary embodiments of gasket 134 include polymer based materials or cellulose based materials. In other exemplary embodiments, the gasket 134 is flexible and less rigid and may become deformed or shrink during a sterilization cycle. In another exemplary embodiment, the gasket 134 can be made out of any type of material that is both flexible, less rigid and is able to maintain its integrity during and after undergoing a sterilization cycle. Example materials of the gasket 134 may be rubber, closed-cell sponge rubber, silicone, cardboard, plastics, medical grade light board, or like material. The rigid sterilization system 100 may also include hinges or clamps 136 for releasably securing and applying pressure between the rigid cover 106 to the base 102 to form a hermetic seal. The hinges or clamps 136 may further include a tamper-evident device 138 to detect whether the hermetic seal has been released after sterilization.

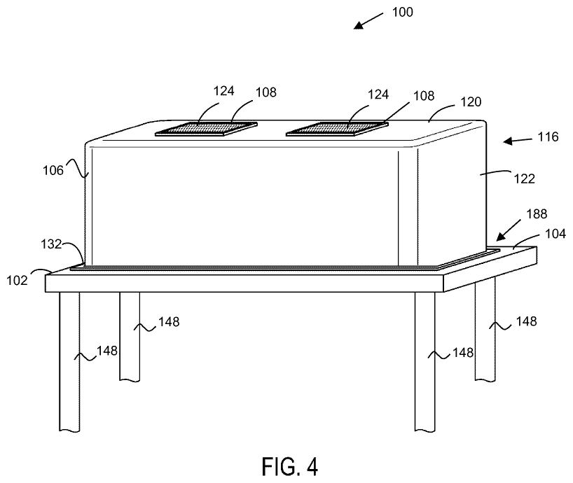

In another exemplary embodiment, as shown in FIG. 4, the base 102 includes a perimetric groove 132 within the surface 104, which groove is dimensioned so that the lower edge 130 of the rigid cover 106 engages the groove 132. Consequently, when pressure is applied to the rigid cover 106, a perimetric seal is formed between the lower edge 130 of the rigid cover 106 and the base 102. In one configuration a gasket is disposed within the groove 132. In another configuration, the gasket is affixed to the lower edge 130 of the rigid cover 106. By “affixed” it is meant that the gasket is permanently affixed, integral or removably affixed to the lower edge 130 of the rigid cover. In either configuration, when the lower edge 130 of the rigid cover 106 is inserted into the groove 132 a perimetric, continuous seal is formed between the rigid cover 106 and the base 102.

The rigid cover 106 may include a top panel 120 and side panels 122 extending from the top panel to form a volume to encompass the object requiring sterilization. In one configuration, the rigid cover 106 encompasses the object requiring sterilization and the sidewalls 112 of the base 102, including the support surface 104. The rigid cover 106, thus, has a tub-like structure wherein the side panels 122 extend downwardly from the top panel 120 towards the base 102. In one configuration, the base 102 does not have a tub-like structure, but rather provides a flat or substantially flat support surface 142 for receiving an object to be sterilized 144. The base 102 may include a rack system affixed to the base 102 for holding the object to be sterilized 144. However, the rack system, such as object separator 114, does not provide sidewalls that extent above the object 144, which therefore, increases the accessibly of the object 144 during removal of the object 144 from the base 102 and reduces possible contamination of the object 144 during the removal of the object 144 from the base 102. The top panel 120 of the rigid sterilization system 100 may include a transparent window for viewing the contents within the rigid sterilization system 100.

As shown in FIGS. 1-7, the main body portion 116 further includes at least one vent port 108. For example, each of the top panel 120 and side panels 122 may include a vent port 108. Alternatively, a vent port 108 may be positioned on each of the side panels 122 or on the top panel 120, but not both. In yet another example embodiment, the top panel 120 and side panels 122 each include multiple vent ports 108. In still a further example embodiment, a vent port 108 is positioned on one, two or three side panels 120. A filter 124 overlies each of the vent ports 108 and forms a sealed interface with the main body portion 116. The filters 124 may be removeably secured to the rigid cover 106 with clamps or hinges. In another exemplary embodiment, the rigid cover 106 includes a vented area comprising a fenestrated grid, holes, gaps, or other grids that allow sterilizing steam to pass through the vented area.

The filter 124 may include a gasket 126 surrounding the filter 124 such that the filter 124 and gasket 126 form a filter cartridge. In a further configuration, the filter cartridge may include a gasket 126 between two filters 124. In another configuration, the filter cartridge may include a gasket 126 affixed to a double layer of filters 124. In yet another configuration, one or two filters are affixed between two gaskets 126. By “affixed” it is meant that the gasket is permanently affixed, integral or removably affixed to the filters. By “filter” it is meant that the material is porous enough to allow the passage of a sterilizing agent, such as steam through its membrane, but also resilient enough to not rip or tear during a sterilizing cycle or during insertion/clamping into a filter holder. In another embodiment, “filter” is porous and less resilient such that the filter can be ripped or torn during use in a sterilizing cycle or with filter holder.

In a configuration, as shown in FIG. 9, the base 102 includes a locking mechanism to releasably lock the base to a base pedestal, legs, or transfer cart 192. The transfer cart 192 may comprise a frame 252, push handles 254, and wheels 256. If the transfer cart 192 is serving as an operating back room table, the transfer cart 192 may optionally not include push handles 254 or a locking mechanism. The transfer cart 192 can be constructed out of any durable material that is strong enough to maintain its shape and form under significant weight and also able to be sterilized. Ideally, transfer cart 192 is constructed out of a steel or aluminum alloy or a combination thereof. It should be understood that transfer cart 192 is merely an exemplary embodiment of a transfer cart 192. In one configuration, the base 102 includes legs 148 that connect to the transfer cart 192. In another configuration, the base 102 is received by legs 148 on the transfer cart 192. Although FIG. 9 shows the configuration shown in FIG. 3 affixed to the transfer cart 192, wherein the rigid cover 106 encompasses the base 102, it should be appreciated that the rigid sterilization system as shown in FIG. 4 may alternatively be affixed to the transfer cart 192. By “affixed” it is meant that the base 102 is permanently affixed, integral or releaseably affixed to the transfer cart 192.

The transfer cart 192 may further include a sled (not shown) having wheels for sliding along transfer cart 192 guiderails when the sled and base 102 are loaded into a sterilizer 10. The transfer cart 192 is rigid enough to maintain the weight of the base 102 and sled or other like sterilizing device or apparatus. In one configuration, the base 102 releaseably locks to the sled via a locking mechanism. The locking mechanism provides a means to removeably affix the base 102 to the transfer cart 192. Exemplary embodiments of the locking mechanism include any means that would allow a base to be securely affixed a transfer cart 192, for example, a sled of the transfer cart 192, such that the base 102 only moves when transfer cart 192 moves. Additionally, the sled may releasably lock to the transfer cart 192 via a locking mechanism, which provides means to removeably affix the sled to the transfer cart 192. Exemplary embodiments of the locking mechanism include any means that would allow a sled to be securely affixed to the transfer cart 192 such that the sled only moves when transfer cart 192 moves. The transfer cart 192 may further include a locking mechanism 262 to releasably lock the transfer cart 192 to the sterilizer 10. Thus, the transfer cart 192 can be locked relative to the sterilizer 10 when the sled and/or base 102 is loaded into the sterilizer 10 to prevent unwanted movement of the transfer cart 192 during the weight transfer of the load from the transfer cart 192 to the sterilizer 10. Exemplary embodiments of the locking mechanisms include clamps, laches, slots, bolts, screws, and the like. In another configuration, the base 102 may be fixedly secured to the transfer cart 192. For example, in one configuration, the base 102 is integral and permanently affixed to the transfer cart 192.

Push handles 254 of transfer cart 192 provide the means for a user to more easily push and pull transfer cart 192. Push handles 254 may be provided on opposite sides of transfer cart 192. Each of the push handles 254 span the width of transfer cart 192. It should be appreciated, that embodiments of push handles 254 include push handles 254 being located on all sides or only on one side of transfer cart 192 along with different configurations.

One advantage of having the base 102, such as an operating room back table, loaded directly onto the transfer cart 192 and then into a sterilizer 10 is that once the sterilization cycle is complete, the base 102 (table) can be loaded back onto the transfer cart 192 and transferred directly to the operating room for use again. The instruments or other object, having already been organized on the tray before sterilization, are ready for use without further processing or organizing. In an alternative configuration, the base 102 and transfer cart 192 are loaded together into a ground-loading sterilizer 10. In this configuration, the transfer cart 192, the base 102, and the instruments contained thereon are sterilized in the ground-loading sterilizer 10. Thus, the base 102 is not separated from the transfer cart 192 during sterilization or during transportation.

As shown in FIG. 10, the rigid sterilization system 100 may include multiple bases 102 arranged on a rack system 160. The rack system 160 in one example embodiment includes a plurality of shelves 162 for receiving bases 102, or bases 102, which are covered and sealed with rigid cover 106. Rack system 160 may include sidewalls 164 and a door 166, wherein a plurality of bases 102 and/or bases 102 may be loaded onto the plurality of shelves when the door is in an open position. In one configuration, the shelves 162 are removeable. In another configuration, the shelves are extendable for ease of receiving a base 102. Typically, a plurality of bases 102, each having rigid cover 106 encompassing and sealing the base 102, are inserted into the rack system 160 on the shelves 162 after the bases 102 sealed with the rigid cover 106 have been sterilized in a sterilizer. Thus, a multitude of bases can be stored in the rack system 160 after processing the bases in a sterilizer. The rack system 160, in one exemplary embodiment, includes wheels 168 to allow the rack system 160 holding a plurality of processed bases 102 to be transported into an operating room.

The present invention contemplates that many changes and modifications may be made. Therefore, while the presently-preferred form of the apparatus and method has been shown and described, and several modifications and alternatives discussed, persons skilled in the art will readily appreciate that various additional changes and modifications may be made without departing from the scope of the invention, as defined and differentiated by the following claims.

1. A rigid sterilization system for an object requiring sterilization in a sterilizer, the rigid sterilization system comprising:
a base having a support surface configured to support an object requiring sterilization;
a rigid cover having a main body and a lower portion, the main body comprising a top panel and side panels extending from the top panel, wherein the top panel and side panels comprise a rigid material configured to hold a selected shape, wherein the rigid cover and the base define a volume encompassing the object requiring sterilization; and
a sealing interface between the lower portion of the rigid cover and the base, the sealing interface configured to form a continuous seal between the lower portion of the rigid cover and a first sealing surface of the base.

2. The rigid sterilization system of claim 1, wherein the rigid cover further comprises a first vent port and a first filter overlying the first vent port and forming a sealed interface with an adjacent portion of the rigid cover.

3. The rigid sterilization system of claim 1, wherein the support surface of the base is a substantially flat surface.

4. The rigid sterilization system of claim 1, wherein the base includes a rack system for holding the object.

5. The rigid sterilization system of claim 1, wherein the base includes a plurality of sides, and wherein the first sealing surface is located on the plurality of sides.

6. The rigid sterilization system of claim 4, further comprising a gasket between the lower portion of the rigid cover and the first sealing surface.

7. The rigid sterilization system of claim 1, wherein the first sealing surface is located on the support surface and wherein the first sealing surface further comprises a gasket within the first sealing surface on the support surface.

8. The rigid sterilization system of claim 1, further comprising a gasket affixed to the lower portion of the rigid cover.

9. The rigid sterilization system of claim 1, wherein the top panel includes a window for viewing contents within the rigid sterilization system.

10. The rigid sterilization system of claim 1, wherein the base is an operating room back table.

11. The rigid sterilization system of claim 1, wherein the rigid cover is selected from the group consisting of heavy gauge aluminum and medical grade plastic material.

12. The rigid sterilization system of claim 1, further comprising a clamp for releasably securing the rigid cover to the base.

13. The rigid sterilization system of claim 2, wherein the first filter overlying the first vent port further comprises a gasket surrounding the first filter, the first filter and gasket forming a filter cartridge, wherein the filter cartridge is removably secured to the main body.

14. The rigid sterilization system of claim 2, wherein the first vent port is located on at least one of the top panel or one of the side panels.

15. The rigid sterilization system of claim 1, wherein the lower portion of the rigid cover overlies the first sealing surface of the base to form the sealed interface between the lower portion of the rigid cover and the base.

16. The rigid sterilization system of claim 1, wherein the base further comprises a groove for receiving a gasket, and wherein the lower portion of the rigid cover further comprises an edge, wherein the edge engages the gasket within the groove for forming a perimetric seal between the edge and the base.

17. The rigid sterilization system of claim 1, further comprising:
a plurality of bases, each base having the rigid cover and the continuous seal between the lower portion of the rigid cover and the first sealing surface; and
a rack having a plurality of shelves, each of the plurality of shelves configured to hold one of the plurality of bases having the rigid cover after sterilization in a sterilizer.

18. The rigid sterilization system of claim 1, wherein the base is configured to affix to a transport cart.

19. A rigid sterilization system for use in a sterilizer for the sterilization of objects requiring sterilization, the system comprising:
a base formed of a material adapted to withstand exposure to a sterilizing agent, the base having (i) a support surface having a portion configured to support the objects requiring sterilization, and (ii) a first sealing surface;
a rigid cover comprising a rigid material configured to hold a selected shape, the cover having a main body portion and a lower portion, wherein the main body portion includes a first vent port and a first filter overlying the first vent port, and wherein the lower portion of the rigid cover is configured to form a sealed interface with the base; and
a continuous seal interface between the first sealing surface of the base and the rigid cover, the continuous seal interface forming a continuous seal between the base and the rigid cover.

20. The rigid sterilization system of claim 19, wherein the support surface of the rigid base is substantially flat.

21. The rigid sterilization system of claim 19, further comprising a gasket between the lower portion of the rigid cover and the first sealing surface.

22. The rigid sterilization system of claim 21, wherein the base further comprises a plurality of sides, and wherein the first sealing surface is located on the plurality of sides, and wherein the gasket is between the lower portion of the rigid cover and the plurality of sides.

23. The rigid sterilization system of claim 21, wherein the first sealing surface is located on the support surface and wherein the gasket is located on one of the first sealing surface and the lower portion of the rigid cover.

24. The rigid sterilization system of claim 19, further comprising:
a plurality of bases, each base having the rigid cover and the continuous seal between the lower portion of the rigid cover and the first sealing surface; and
a rack having a plurality of shelves, each of the plurality of shelves configured to hold one of the plurality of bases having the rigid cover after sterilization in a sterilizer.

25. The rigid sterilization system of claim 19, wherein the base is configured to affix to a transport cart.
